(19) United States
(12) Patent Application Publication
(10) Pub. No.: US 2017/0325939 A1
(43) Pub. Date: Nov. 16, 2017

(54) SUTURING RINGS FOR PROSTHETIC HEART VALVES

(71) Applicants: Sujay Kumar Shad, New Delhi (IN); Manish Vaishya, Valencia, CA (US)

(72) Inventors: Sujay Kumar Shad, New Delhi (IN); Manish Vaishya, Valencia, CA (US)

(21) Appl. No.: 15/151,439

(22) Filed: May 10, 2016

(51) Int. Cl. A61F 2/24

(52) U.S. Cl. CPC A61F 2/2409; A61F 2240/001

(57) ABSTRACT

The present subject matter describes a suturing rings and method of manufacture of suturing rings for prosthetic heart valves. A plurality of suture holes are formed along a length of the metal sheet by performing punching or similar operations. The metal sheet has a substantially constant thickness throughout the length of the metal sheet. The rectangular metal sheet is rolled along the length until two ends of the metal sheet abut with each other. The two ends of the metal sheet are welded to form a metallic tube. The metallic tube is reshaping using a forming tool to obtain a suturing ring having an inner surface with a substantially convex profile. The suturing ring has an inner diameter at an inner peripheral edge at a first open end and an inner diameter at an inner peripheral edge at a second open end greater than an inner diameter at a mid-circumferential plane.

### TECHNICAL FIELD

The present subject matter relates, in general, to suturing rings, and in particular to suturing rings for prosthetic heart valves and methods of manufacturing such suturing rings.

### BACKGROUND

Valves perform important physiological functions in a human heart by controlling the direction and volume of blood flow through the heart. Typically, there are four valves in the human heart, viz., tricuspid valve, pulmonic valve, mitral valve, and aortic valve. These valves regulate and maintain the blood flow to and from the heart. The aortic and pulmonic valves regulate flow form the ventricles into the aorta and pulmonary arteries, respectively. The mitral and tricuspid valves regulate flow from the left and right atria, respectively, into the ventricles. The human heart can suffer from various valvular diseases, which then broadly progress to stenosis or insufficiency. This can further result in significant malfunctioning of the heart and may require replacement of a native/natural valve in the heart with an artificial heart valve, also called a prosthetic heart valve. The prosthetic heart valve, like a bioprosthetic or mechanical heart valve, can be implanted in the human heart to replicate the function of the natural heart valve.

### BRIEF DESCRIPTION OF DRAWINGS

The detailed description is described with reference to the following figures. It should be noted that the description and figures are merely examples of the present subject matter and are not meant to represent the subject matter itself.

FIG. 1a illustrates a perspective view of a suturing ring for a prosthetic heart valve, in accordance with an example implementation of the present subject matter.

FIG. 1b illustrates a cross-sectional view of the suturing ring of FIG. 1a, in accordance with an example implementation of the present subject matter.

FIG. 2a illustrates a method of manufacturing the suturing ring of FIG. 1a, in accordance with an example implementation of the present subject matter.

FIG. 2b illustrates a sectional view of a first mandrel used for reshaping a metallic tube for manufacturing the suturing ring of FIG. 1a, according to an example implementation of the present subject matter.

FIG. 3 illustrates a method of manufacturing the suturing ring of FIG. 1a, in accordance with an example implementation of the present subject matter.

FIG. 4a illustrates a perspective view of a suturing ring for a prosthetic heart valve, in accordance with an example implementation of the present subject matter.

FIG. 4b illustrates a cross-sectional view of the suturing ring of FIG. 4a, in accordance with an example implementation of the present subject matter.

FIG. 5a illustrates a perspective view of a suturing ring for a prosthetic heart valve, in accordance with an example implementation of the present subject matter.

FIG. 5b illustrates a cross-sectional view of the suturing ring of FIG. 5a, in accordance with an example implementation of the present subject matter.

### DETAILED DESCRIPTION

Prosthetic heart valves, hereinafter interchangeably referred to as prosthetic valves, can be of different types such as, single leaflet valves, bi-leaflet valves, caged ball valves, and the like. Generally, a bi-leaflet prosthetic valve includes a valve housing, and two semicircular leaflets are mounted within the valve housing through struts. The semicircular leaflets can rotate about the struts between an open position and a closed position of the prosthetic valve to regulate flow of blood though the prosthetic valve. The prosthetic valve is stitched, or sutured, to native tissue with the help of suturing rings that form the valve housing.

Conventionally, a suturing ring includes a fabric layer wrapped around the suturing ring. Suture-threads are passed though the fabric layer to stitch the valve with the native tissue. The fabric layer formed from woven fibers has a certain degree of porosity that encourages growth of tissue and infectious seeding within the pores of the fabric layer and thereby leads to medical conditions post implant of the prosthetic valve.

Further, it may be noted that the fabric layer around the suturing ring has a substantial thickness in order to ensure proper stitching of the valve with the native tissue. Since, the outer diameter of the valve is substantially fixed depending on the aortic diameter where the valve is to be implanted, the thickness of the fabric layer in addition to the thickness of the valve housing/suturing ring puts a limitation on the extent to which the inner diameter of the valve can be increased. A smaller inner diameter limits the effective orifice area of the valve through which blood can flow, which may lead to a patient's prosthesis mismatch that may create a large pressure gradient of blood flowing through the valve. As a result, the heart may have to work harder, which may cause heart muscles to become more muscular leading to a medical condition known as left ventricle hypertrophy.

There may be another conventional suturing ring that does not have a fabric layer around the suturing ring. Such suturing rings have an annular cylindrical structure having an inner surface which forms a boundary of the effective orifice area of the valve and an outer surface which abuts with native tissue walls of the patient when the valve is implanted in the patient's body. The outer surface of the conventional suturing ring has a substantially curved profile while the inner surface has a substantially planar or straight profile. Such suturing rings have suture-receiving passages or holes formed in the suturing ring. Suture-threads are passed through the suture-receiving passages to stitch the valve with the native tissue. Since, this conventional valve eliminates the use of the fabric layer, chances of tissue growth and infectious seeding of the fabric layer are eliminated. Also, as there is no fabric layer, the inner diameter of the valve may be increased beyond the inner diameter of the valve having a suturing ring with a fabric layer.

However, in the conventional suturing ring, due to the planar surface profile of the inner surface a substantial non-uniformity is introduced in the flow path of blood flowing through the valve when implanted in a human heart. Particularly, an L-shaped profile formed at an inlet of the suturing ring through which the blood enters the valve and at an outlet of the suturing ring through which the blood leaves the valve introduces an abrupt obstruction in the flow path of the blood. The abrupt obstruction in the flow path creates substantial turbulence in the flow of blood through the valve particularly at the inlet and the outlet. The turbulence in the flow of blood through the valve may result in medical complications, such as thrombosis, and may contribute to pannus formation in the patient.

Generally, in the conventional suturing ring without the fabric layer, the suture-receiving passages are fabricated by drilling through-holes. Since the working dimensions of the conventional suturing ring is of the order of a few millimeters, fabrication of the suture-receiving passages is difficult. In addition to this, during the fabrication of the suture-receiving passages by drilling of through-holes there are chances of fracturing or damaging the conventional suturing ring, particularly at the first open end and the second open end. Fracturing of the conventional suturing ring may lead to valve instability, para-valvular regurgitation, and increases the risk of embolization of the valve.

To avoid such fractures or damages, a substantial thickness is to be maintained between the inner surface and the outer surface of the conventional suturing ring. Since the outer diameter of the conventional suturing ring is substantially fixed depending on the aortic diameter where the valve is to be implanted, the substantial thickness between the inner surface and the outer surface puts a limitation on the extent to which the inner diameter of the conventional suturing ring can be increased. A smaller inner diameter of the conventional suturing ring limits the effective orifice area for blood flow through the valve which may lead to a patient prosthesis mismatch. A severe mismatch can increase the chances of mortality by a factor of 10 in the long run. Also, the limited effective orifice area for flow of blood through the valve 200 may pose risks of medical conditions, such as left ventricle hypertrophy, especially in smaller heart valves, such as heart valves for infants.

The present subject matter describes suturing rings for prosthetic heart valves and methods of manufacturing the suturing rings. The suturing rings of the present subject matter eliminate use of the fabric layer around the suturing ring, provide a larger effective orifice area for blood flow through prosthetic heart valves as compared to the conventional valves, provide blood flow with reduced turbulence and improved hemodynamics, and thereby prevent a variety of medical conditions, such as, thrombosis, prosthesis mismatch, left ventricle hypertrophy, etc. Further, the methods of manufacturing the suturing rings of the present subject matter provide a simple way of manufacturing the suturing rings of the present subject matter with reduced machining requirements and minimized loss of raw materials.

In an example implementation, the suturing ring of the present subject matter forms a valve housing of a prosthetic heart valve. The suturing ring has an inner surface having a substantially convex profile. The convex profile is such that the suturing ring has an inner diameter at an inner peripheral edge at a first open end (e.g., inlet) of the suturing ring and at an inner peripheral edge at a second open end (e.g., outlet) of the suturing ring greater than an inner diameter at a mid-circumferential plane of the suturing ring. The suturing ring has a substantially constant annular thickness from the first open end to the second open end of the suturing ring.

Since the suturing ring of the present subject matter has an inner surface with a substantially convex profile, the blood flowing through the valve has less turbulence and a better hemodynamic flow pattern. Thus, chances of thrombosis are reduced.

Further, in an example implementation, the inner surface of the suturing ring has a planar or straight portion. The planar portion is substantially perpendicular to the mid-circumferential plane of the suturing ring and extends up to a fixed distance on either side of the mid-circumferential plane. Beyond the planar portion, the inner surface of the suturing ring has a curved surface profile bending outwards towards the first open end and the second open end. The planar portion facilitates easier attachment of valve leaflets within the suturing ring that forms the valve housing of the prosthetic valve.

Further, in an example implementation, the suturing ring of the present subject matter has suture holes, also referred to as suture-receiving passages, that pass from the inner surface to the outer surface of the suturing ring. The suture-receiving passages in the suturing ring of the present subject matter are easier to fabricate and the chances of fracturing or damaging the suturing ring are reduced.

Further, the design of the suturing ring of the present subject matter and the suture-receiving passages from the inner surface to the outer surface enables reduction in the thickness between the inner and outer surfaces of the suturing ring. This allows a valve with the suturing ring of the present subject matter to have a larger inner diameter than the inner diameter in conventional prosthetic heart valves. The larger inner diameter results in increasing the effective orifice area of the prosthetic valve that effectuates an increase in the volume of blood flow through the prosthetic valve, thereby, minimizing the chances of patient prosthesis mismatch in even the smallest heart valves, such as heart valves for infants.

A method of manufacturing a suturing ring for a prosthetic heart valve according to an example implementation of the present subject matter is described. A plurality of suture-receiving passages or holes are formed through a rectangular metal sheet along a length of the metal sheet by performing punching or similar operations on the metal sheet. The metal sheet has a substantially constant thickness throughout the length of the metal sheet. The rectangular metal sheet is rolled along the length until two ends (breadth-wise) of the metal sheet abut with each other. The two ends of the metal sheet are welded to form a metallic tube. The metallic tube is reshaped using a forming tool to obtain a suturing ring having an inner surface with a substantially convex profile. The metallic tube is reshaped is such that an inner diameter at an inner peripheral edge at a first open end of the suturing ring and an inner diameter at an inner peripheral edge at a second open end of the suturing ring is greater than an inner diameter at a mid-circumferential plane of the suturing ring.

The method of the present subject matter is simpler as it eliminates a variety of machining processes or deep drawing, otherwise conventionally used for manufacturing of suturing rings. Elimination of these machining processes also facilitate in providing suturing rings with a reduced annular thickness as compared to the conventional suturing rings. This increases the effective orifice area and reduces chances of blood clot.

Further, reshaping the metallic tube using the forming tool at normal temperature, followed by deburring and annealing processes allows for cold forming of the suturing ring of the present subject matter. Due to cold forming, additional strength is provided to the material of the suturing ring of the present subject matter.

Additionally, forming the plurality of suture-receiving passages or suture holes through the rectangular metal sheet all at once is simpler, instead of drilling each passage one by one through the suturing ring, as in the conventional method. This also reduces a substantial amount of manufacturing time and eliminates high machining costs. All of these, further facilitate in reduction of manufacturing costs of the suturing rings of the present subject matter.

The above mentioned implementations are further described herein with reference to the accompanying figures. It should be noted that the description and figures relate to exemplary implementations, and should not be construed as a limitation to the present subject matter. It is also to be understood that various arrangements may be devised that, although not explicitly described or shown herein, embody the principles of the present subject matter. Moreover, all statements herein reciting principles, aspects, and embodiments of the present subject matter, as well as specific examples, are intended to encompass equivalents thereof.

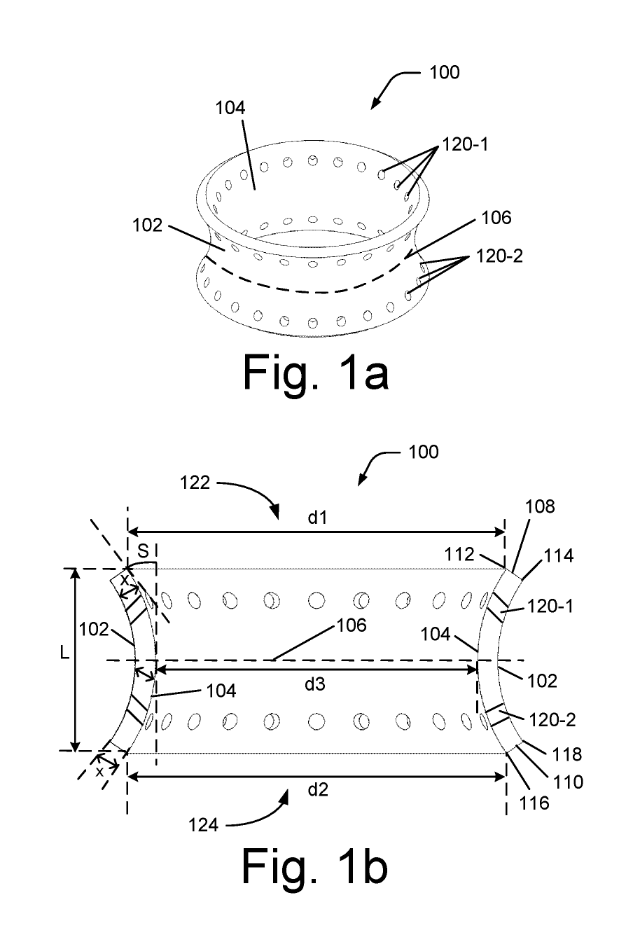

FIG. 1a illustrates a perspective view of a suturing ring 100 for a prosthetic heart valve, in accordance with an example implementation of the present subject matter. The suturing ring 100 may form a valve housing of the prosthetic heart valve. The suturing ring 100 has an annular structure having an outer surface 102 and an inner surface 104. The inner surface 104 has a substantially convex profile and the outer surface 102 has a substantially concave profile. When the prosthetic valve is surgically implanted within the human body, the outer surface 102 abuts with native tissues in the human body and the inner surface 104 comes in contact with blood flowing through the prosthetic valve.

FIG. 1a depicts a boundary 106 of a mid-circumferential plane of the suturing ring 100. The suturing ring 100 is symmetrical about the mid-circumferential plane, as shown in FIG. 1a. In an example implementation, the suturing ring may be asymmetrical (not shown) about the mid-circumferential plane.

FIG. 1b illustrates a cross-sectional view of the suturing ring 100, in accordance with an example implementation of the present subject matter. In the cross-sectional view of the suturing ring 100, the curved line referenced as 104 represents the inner surface of the suturing ring 100, and the curved line referenced as 102 represents the outer surface of the suturing ring 100. The suturing ring 100 has a first open end 122 and a second open end 124. In an example implementation, the first open end 122 and the second open end 124 may be separated by a linear distance, referenced as L, in a range of about 4.5 mm to about 10.5 mm.

The suturing ring 100 has a first end surface 108 at the first open end 122 and a second end surface 110 at the second open end 124. The inner surface 104 meets the first end surface 108 at an inner peripheral edge 112 at the first open end 122, and the outer surface 102 meets the first end surface 108 at an outer peripheral edge 114 at the first open end 122. Similarly, the inner surface 104 meets the second end surface 110 at an inner peripheral edge 116 at the second open end 124 and the outer surface 102 meets the second end surface 110 at an outer peripheral edge 118 at the second open end 124.

Due to the substantially convex profile of the inner surface 104, an inner diameter, referenced as d1, at the inner peripheral edge 112 at the first open end 122 and an inner diameter, referenced as d2, at the inner peripheral edge 116 at the second open end 124 are greater than the inner diameter, referenced as d3, at the mid-circumferential plane of the suturing ring 100. In an example implementation, the inner diameter of the suturing ring 100 gradually increases from d3 at the mid-circumferential plane to d1 at the first open end 122 and to d2 at the second open end 124. The inner diameter d1 at the inner peripheral edge at the first open end 122 and the inner diameter d2 at the inner peripheral edge at the second open end 124 are identical. In an example implementation, the inner diameter d3 of the suturing ring 100 at the mid-circumferential plane may be in a range from about 12 mm to about 32 mm, and the inner diameter d1 and d2 of the suturing ring 100 at the inner peripheral edge at the first open end 122 and at the inner peripheral edge at the second open end 124 may be in a range from about 15 mm to about 35 mm.

The suturing ring 100 has a substantially constant annular thickness ‘x’ from the first open end 122 to the second open end 124, as shown in FIG. 1b. The annular thickness may be defined as a distance between the inner surface 104 and the outer surface 102 of the suturing ring 100. In an example implementation, the constant annular thickness of the suturing ring 100 is in a range from about 0.3 mm to about 1.5 mm.

As shown in FIG. 1a, the suturing ring 100 has suture holes 120-1 and 120-2, hereinafter also referred to as suture-receiving passages 120-1 and 120-2, from the inner surface 104 to the outer surface 102 on either sides of the mid-circumferential plane. A first set of suture-receiving passages 120-1 are present between the mid-circumferential plane and the inner peripheral edge 112 at the first open end 122, and a second set of suture-receiving passages 120-2 are present between the mid-circumferential plane and the inner peripheral edge 116 at the second end. The plurality of suture-receiving passages 120-1 and 120-2 may be collectively referred to as suture-receiving passages 120. Suture-threads may be passed through the suture-receiving passages 120. The suture-threads enable stitching the prosthetic valve to the native human tissue at the position of implant of the prosthetic valve, such as the aortic annulus, lying between the aorta and the left ventricle of the human heart.

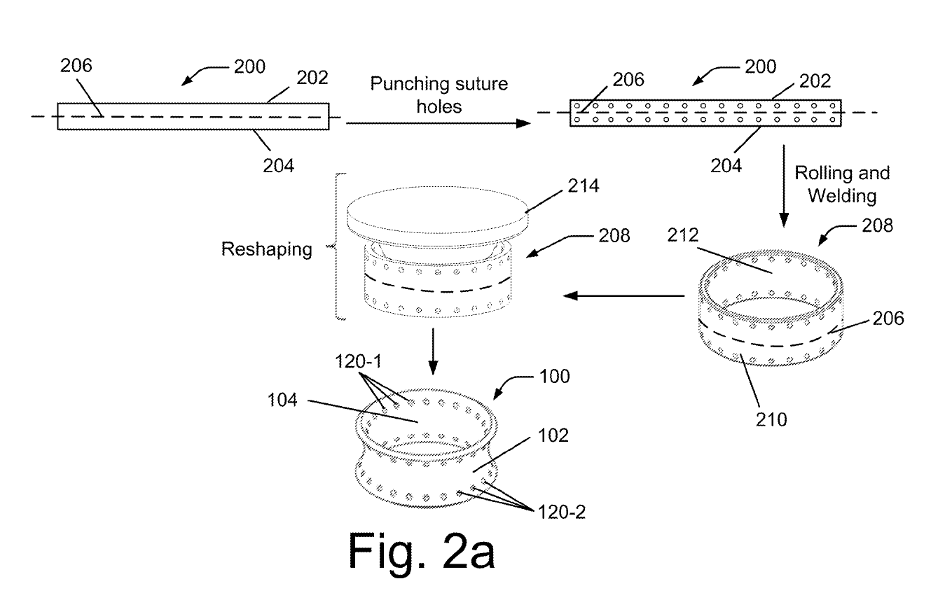

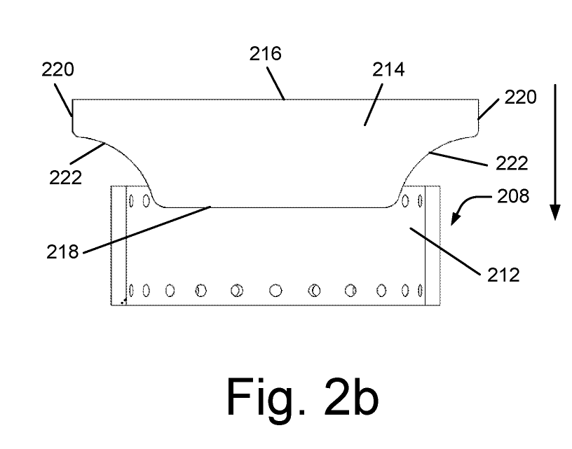

FIG. 2a illustrates a method of manufacturing the suturing ring 100 of FIG. 1a, in accordance with an example implementation of the present subject matter. Different stages of manufacturing the suturing ring 100 are illustrated in FIG. 2a.

In an example implementation, a metal sheet 200 is obtained by metal forming processes, such as rolling and shearing. The metal sheet 200 is formed from implantable grade bio-compatible metals, such as titanium. The metal sheet 200 is rectangular in shape and has a length substantially equal to a perimeter of a suturing ring to be formed, and has a width substantially equal to the width of the suturing ring to be formed. The metal sheet 200 has a substantially constant thickness throughout the length of the metal sheet 200. The rectangular metal sheet 200 has a first linear edge 202 parallel to a second linear edge 204 along the length of the metal sheet 200. A line 206 along the length of the metal sheet 200 and in between the first linear edge 202 and the second linear edge 202 represents a central longitudinal axis of the metal sheet 200 which divides the metal sheet into two symmetrical halves.

A plurality of suture holes are formed along the length of the rectangular metal sheet 200 by performing various operations on the metal sheet 200, such as punching, drilling, boring and the like. The plurality of suture holes would form the suture-receiving passages 120-1 and 120-2 of the suturing ring 100.

In an example implementation, the plurality of suture holes are punched through the metal sheet 200 at a uniform distance from each other, as shown. A first set of suture holes are formed in the metal sheet 200 between the central longitudinal axis 206 and the first linear edge 202 and a second set of suture holes are formed in the metal sheet 200 between the central longitudinal axis 206 and the second linear edge 204. In an example implementation, the first set and the second set of suture holes may be punched in a single operation.

After the suture holes are formed, the metal sheet 200 is rolled along the length until two ends of the metal sheet abut with each other. In an example implementation, the metal sheet 200 may be rolled by passing the metal sheet 200 through one or more pairs of rollers. Rolling may eliminate any non-uniformities on the surface of the metal sheet 200. Subsequently, the two ends of the metal sheet 200 are welded to form a metallic tube 208. The metallic tube 208 has an annular shape with an outer surface 210 and an inner surface 212, as shown in FIG. 2a. It may be noted that the outer surface 210 and the inner surface 212 have a substantially planar profile.

The metallic tube 208 is then reshaped by using a forming tool to obtain the suturing ring 100 that has an inner surface with a substantially convex profile. In an example implementation, the forming tool includes machining tools, such as mandrels that are used for performing metallurgical operations like forming and reshaping. In an example implementation, the metallic tube 208 is reshaped using cold forming techniques at room temperature. Cold forming provides additional strength to the metal and thereby improves durability of the suturing ring that is formed.

As shown in FIG. 2a, a first mandrel 214 with an axisymmetric shape that directly corresponds to the inner surface profile of the suture ring, is pressed against the metallic tube 208 from a first open end of the metallic tube 208 to exert an outward force on the inner surface 212 of the metallic tube 208. Similarly, a second mandrel (not shown) with an axisymmetric shape that directly corresponds to the inner surface profile of the suture ring is pressed against the metallic tube 208 from a second open end opposite to the first open end of the metallic tube 208 to exert an outward force on the inner surface 212 of the metallic tube 208. The force is exerted on the inner surface 212 of the metallic tube 208 by applying an appropriate load on the metallic tube 208 which depends upon a number of design and process parameters. In an example implementation, the load exerted by the first mandrel 214 and the second mandrel is in a range of about 5 tonnes to about 10 tonnes. In an example implementation, the first mandrel and the second mandrel has identical diameters to obtain the suturing ring 100 that is symmetrical about the mid-circumferential plane. The procedure of reshaping the metallic tube 208 using the mandrels is further described with reference to FIG. 2b.

Upon reshaping the metallic tube 208, the substantially convex profile of the inner surface 104 of the suturing ring 100 is obtained. After reshaping, in an example implementation, metallurgical operations, such as annealing and deburring may be performed for finishing of the metal surface and for smoothening out sharp edges. Subsequently, the suture ring may be coated with Pyrolytic Carbon.

The method of manufacturing the suturing ring 100 as described above has minimal material wastage and reduced machining costs. Also, forming the multiple suture-receiving passages, all at once, through the metal sheet reduces the overall manufacturing time as compared to the manufacturing time in a conventional procedure where individual suture-receiving passages are drilled through the suturing ring.

FIG. 2b illustrates a sectional view of the first mandrel 214 used for reshaping the metallic tube 208 for manufacturing the suturing ring 100 of FIG. 1a, according to an example implementation of the present subject matter. As shown in FIG. 2b, the first mandrel 214 has a first flat surface 216 and a second flat surface 218. The first flat surface 216 and the second flat surface 218 have a circular perimeter along their edges and the second flat surface 218 has a smaller surface area than the first flat surface 216.

A planar surface, referenced by the line(s) 220, extends perpendicularly from an edge of the first flat surface 216 towards the second flat surface 218. The planar surface 220 and the second flat surface 218 are separated by a curved surface, referenced by the curved line(s) 222 in FIG. 2b. The curved surface 222 has a concave surface profile that corresponds to the inner surface profile of the suture ring 100 to be formed.

In an example implementation, for reshaping the metallic tube 208, the first mandrel 214 is pressed against an open end of the metallic tube 208. As shown in FIG. 2b, the arrow indicates the direction in which the first mandrel 214 is pressed against the metallic tube 208. Thus, the curved surface 222 of the first mandrel 214 exerts an outward force on the inner surface 212 of the metallic tube 208. As a result of the outward force, the inner surface 212 bends outwards to form a curved surface profile. In a similar fashion, a second mandrel (not shown) may be pressed against the other open end of the metallic tube 208 for reshaping. After reshaping, the suturing ring 100 with the inner surface 104 having a convex surface profile is obtained.

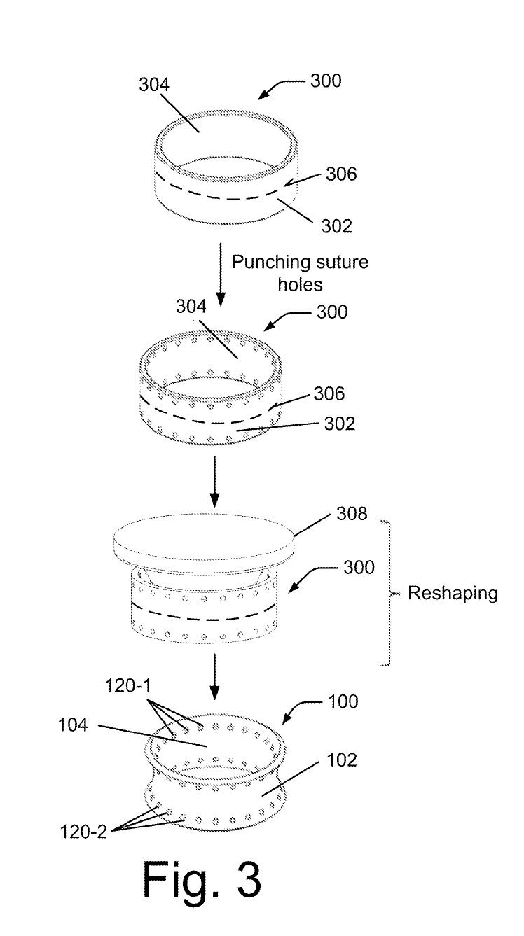

FIG. 3 illustrates a method of manufacturing the suturing ring 100 of FIG. 1a, in accordance with an example implementation of the present subject matter. Different stages of manufacturing the suturing ring 100 are illustrated in FIG. 3.

A metallic tube 300 is obtained by metal forming processes, such as forging and shaping. The metal tube is formed from, for example, titanium. In an example implementation, the metallic tube 300 may be formed by rolling a rectangular metal sheet of substantially constant thickness along a length of the metal sheet until two ends of the metal sheet abut with each other. The two abutted ends of the metal sheet may be welded together to form the metallic tube 300. Alternatively, the metallic tube 300 may be formed seamlessly by a metal drawing operation. These tubes are generally standard parts and may be available in pre-existing tubular form.

The metallic tube 300 has an annular shape with an outer surface 302 and an inner surface 304, as shown in FIG. 3. FIG. 3 depicts a boundary 306 of a mid-circumferential plane of the metallic tube 300. The metallic tube 300 is symmetrical about the mid-circumferential plane, as shown in FIG. 3. The metallic tube 300 has a first open end on one side of the mid-circumferential plane and a second open end opposite to the first open end on the other side of the mid-circumferential plane. A first end surface (not shown) is present at the first open end and a second end surface (not shown) is present at the second open end. The inner surface 304 meets the first end surface at an inner peripheral edge (not shown) at the first open end and the outer surface 302 meets the first end surface at an outer peripheral edge (not shown) at the first open end. Similarly, the inner surface 304 meets the second end surface at an inner peripheral edge (not shown) at the second open end and the outer surface 302 meets the second end surface at an outer peripheral edge (not shown) at the second open end.

A plurality of suture holes are formed in the metallic tube 300 by performing various operations, such as, punching, machining, boring, and drilling. In an example implementation, a first set of suture holes may be formed in the metallic tube 300 between the mid-circumferential plane of the metallic tube 300 and the inner peripheral edge at the first open end. A second set of suture holes may be formed in the metallic tube 300 between the mid-circumferential plane of the metallic tube 300 and an inner peripheral edge at the second open end of the metallic tube. The plurality of suture holes would form the suture-receiving passages 120-1 and 120-2 of the suturing ring 100.

The metallic tube 300 is then reshaped by using a forming tool to obtain a suturing ring 100 that has an inner surface 104 with a substantially convex profile. In an example implementation, the forming tool includes machining tools, such as mandrels that are used for performing metallurgical operations like forming and reshaping.

As shown in FIG. 3, a first mandrel 308 with an axisymmetric shape that directly corresponds to the inner surface profile of the suture ring is pressed against the metallic tube 300 from the first open end of the metallic tube 300 to exert an outward force on the inner surface 304 of the metallic tube 300. Similarly, a second mandrel (not shown) with an axisymmetric shape that directly corresponds to the inner surface profile of the suture ring is pressed against the metallic tube 300 from the second open end of the metallic tube 300 to exert an outward force on the inner surface 304 of the metallic tube 300. The procedure of reshaping the metallic tube 300 may be similar to the procedure of reshaping the metallic tube 208 using the first and second mandrels, as described earlier with reference to FIG. 2b.

Upon reshaping the metallic tube 300, the suturing ring 100 with the inner surface 104 having the substantially convex profile is formed. In an example implementation, the first mandrel and the second mandrel have identical diameters to obtain the suturing ring 100 that is symmetrical about the mid-circumferential plane. Subsequently, the suturing ring may be coated with Pyrolytic Carbon.

The suturing ring 100 (as shown in FIGS. 1a and 1b) obtained on completion of the manufacturing procedures illustrated through FIGS. 2 and 3 has an uniform annular thickness from the top end to the bottom end of the suturing ring 100. Further, referring to FIG. 1(b), an inner diameter d1 of the suturing ring 100 at an inner peripheral edge 112 at a first open end and an inner diameter d2 of the suturing ring 100 at an inner peripheral edge 116 at a second open end is greater than an inner diameter d3 of the suturing ring 100 at a mid-circumferential plane.

The procedures of manufacturing a suturing ring, as described in FIGS. 2 and 3 may also be used to manufacture a suturing ring that is asymmetrical about the mid-circumferential plane. For manufacturing the suturing ring asymmetrical about the mid-circumferential plane, the first mandrel and the second mandrel used for reshaping has non-identical diameters with respect to each other.

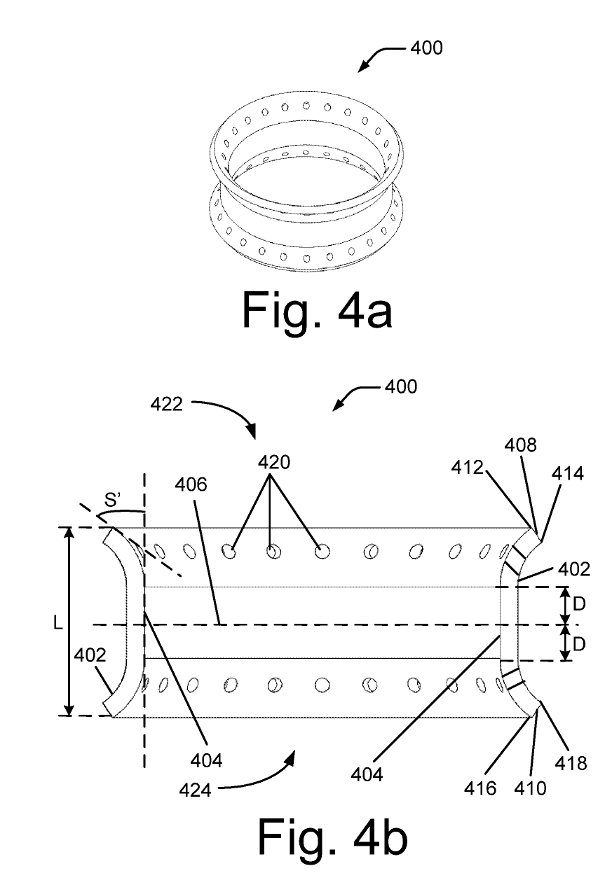

FIG. 4a illustrates a perspective view of a suturing ring for a prosthetic heart valve, in accordance with an example implementation of the present subject matter. FIG. 4b illustrates a cross-sectional view of the suturing ring of FIG. 4a, in accordance with an example implementation of the present subject matter.

Referring to FIG. 4b, the line referenced as 402 represents an outer surface of the suturing ring 400, and the line referenced as 404 represents an outer surface of the suturing ring 400. The inner surface 404 and the outer surface 402 has a substantially convex profile. FIG. 4b depicts a boundary 406 of a mid-circumferential plane of the suturing ring 400. The suturing ring 400 is symmetrical about the mid-circumferential plane.

The suturing ring 400 has a first open end 422 on one side of the mid-circumferential plane and a second open end 424 opposite to the first open end 422 on the other side of the mid-circumferential plane. The inner surface 404 of the suturing ring 400 has a planar or straight portion substantially perpendicular to the mid-circumferential plane. Between the mid-circumferential plane and the first open end 422, the planar portion of the inner surface 404 extends up to a first distance from the mid-circumferential plane towards the first open end 422. Beyond the first distance from the mid-circumferential plane, the inner surface 404 bends outwards towards the first open end 422. Between the mid-circumferential plane and the second open end 424, the planar portion of the inner surface 404 extends up to a second distance from the mid-circumferential plane towards the second open end. Beyond the second distance from the mid-circumferential plane, the inner surface 404 bends outwards towards the second open end 424. In the suturing ring 400 as shown in FIG. 4b, the first distance and the second distance are identical and are referenced as D. The planar or straight portion of the inner surface 404 facilitates in simpler attachment of valve leaflets within the suturing ring 400 which forms as a valve housing for a prosthetic heart valve.

A first end surface 408 is present at the first open end 422 and a second end surface 410 is present at the second open end 424. The inner surface 404 meets the first end surface 408 at an inner peripheral edge 412 at the first open end 422 and the outer surface 402 meets the first end surface 408 at an outer peripheral edge 414 at the first open end 422. Similarly, the inner surface 404 meets the second end surface 410 at an inner peripheral edge 416 at the second open end 424 and the outer surface 402 meets the second end surface 410 at an outer peripheral edge 418 at the second open end 424. The suturing ring 400 has a substantially uniform annular thickness from the first open end 422 to the second open end 424. In an example implementation, the annular thickness is in a range of about 0.3 mm to about 1.5 mm.

In an example implementation, the inner peripheral edge 412 at the first open end 422 and the inner peripheral edge 416 at the second open end 424 may be separated by a linear distance denoted by L in FIG. 4b. The linear distance L shown in FIG. 4b is similar to that shown in FIG. 1b, and may be understood to be the shortest distance between the inner peripheral edge 412 at the first open end 422 and the inner peripheral edge 416 at the second open end 424. The linear distance L maybe in a range from about 4.5 mm to about 10.5 mm.

Further, as shown, the inner surface 404 of the suturing ring 400 at the inner peripheral edge 412 at the first end has an angle of slope S with the vertical line. In an example implementation, the angle of slope ranges from 10 degrees to 80 degrees. It may be noted the suturing ring 400 has suture holes or suture-receiving passages 420, similar to the suture-receiving passages 120 of the suturing ring 100 as shown in FIGS. 1a and 1b.

The suturing ring 400 may be manufactured by following a procedure similar to the procedures for manufacturing the suturing ring 100 as described in FIGS. 2 and 3. In an example implementation, during manufacturing of the suturing ring 400, for reshaping a metallic tube to obtain the suturing ring 400 with a substantially convex inner surface, a mandrel, such as the mandrel 214, as shown in FIG. 2, or the mandrel 308, as shown in FIG. 3, may be used. The mandrel is pressed against the metallic tube such that the mandrel does not reach up to the mid-circumferential plane of the metallic tube. This may be achieved by reducing the pressure at which the mandrel is pressed against the metallic tube or by increasing the diameter of the mandrel substantially larger than the diameter of the metallic tube at the mid-circumferential plane. Thus, after reshaping, a portion of the inner surface on either side of the mid-circumferential plane remains planar or straight and substantially perpendicular to the mid-circumferential plane. The inner surface extending beyond the planar portions on either side of the mid-circumferential plane has a substantially curved profile.

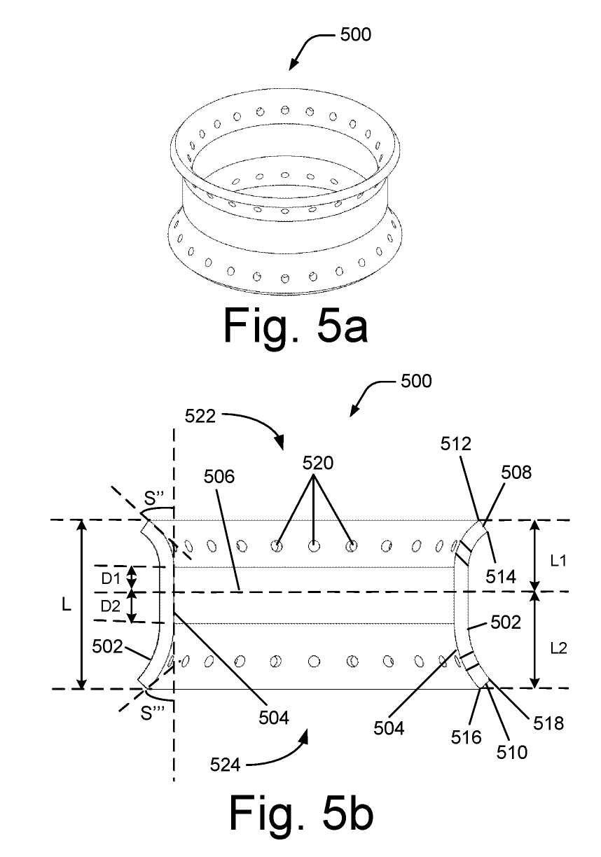

FIG. 5a illustrates a perspective view of a suturing ring 500 for a prosthetic heart valve, in accordance with an example implementation of the present subject matter. FIG. 5b illustrates a cross-sectional view of the suturing ring of FIG. 5a, in accordance with an example implementation of the present subject matter.

Referring to FIG. 5b, the line referenced as 502 represents an outer surface of the suturing ring 500, and the line referenced as 504 represents an outer surface of the suturing ring 500. The inner surface 504 and the outer surface 502 has a substantially convex profile. FIG. 5b depicts a boundary 506 of a mid-circumferential plane of the suturing ring 500.

The suturing ring 500 has a first open end 522 on one side of the mid-circumferential plane and a second open end 524 opposite to the first open end 522 on the other side of the mid-circumferential plane. The suturing ring 500 has a first end surface 508 at the first open end 522 and a second end surface 510 at the second open end 524. The inner surface 504 meets the first end surface 508 at an inner peripheral edge 512 at the first open end 522 and the outer surface 502 meets the first end surface 508 at an outer peripheral edge 514 at the first open end 522. Similarly, the inner surface 504 meets the second end surface 510 at an inner peripheral edge 516 at the second open end 524 and the outer surface 502 meets the second end surface 510 at an outer peripheral edge 518 at the second open end 524.

In an example implementation, the inner peripheral edge 512 at the first open end 522 and the inner peripheral edge 516 at the second open end 524 may be separated by a linear distance denoted by L in FIG. 5b. The linear distance L shown in FIG. 5b is similar to that shown in FIG. 4b, and may be understood to be the shortest distance between the inner peripheral edge 512 at the first open end 522 and the inner peripheral edge 516 at the second open end 524. The linear distance L maybe in a range from about 4.5 mm to about 10.5 mm.

The suturing ring 500 is asymmetrical about the mid-circumferential plane. The inner peripheral edge 512 at the first open end 522 is at a linear distance L1 from the mid-circumferential plane and the inner peripheral edge 516 at the second open end 524 is at a linear distance L2 from the mid-circumferential plane. It may be noted that the linear distance L1 and the linear distance L2, are different with respect to each other.

The inner surface 504 of the suturing ring 500 has a planar or straight portion substantially perpendicular to the mid-circumferential plane. Between the mid-circumferential plane and the first open end 522, the planar portion of the inner surface 504 extends up to a first distance, referenced as D1, from the mid-circumferential plane towards the first open end 522. Beyond the first distance D1 from the mid-circumferential plane, the inner surface 504 bends outwards towards the first open end 522. Between the mid-circumferential plane and the second open end 524, the planar portion of the inner surface 504 extends up to a second distance, referenced as D2, from the mid-circumferential plane towards the second open end 524. Beyond the second distance D2 from the mid-circumferential plane, the inner surface 504 bends outwards towards the second open end 524. In the suturing ring 500 as shown in FIG. 5b, the first distance D1 and the second distance D2 are non-identical with respect to each other. In an example implementation, the first distance D1 is in a range of about 0.5 mm to about 5.5 mm and the second distance D2 is in a range of about 0.5 mm to about 5.5 mm. In an example implementation, the planar portion of the inner surface 504 is in a range of about 1 mm to about 6 mm. The planar portion of the inner surface 504 facilitates simpler attachment of valve leaflets within the suturing ring 500 which forms a valve housing of a prosthetic heart valve.

Further, as shown, the inner surface 504 of the suturing ring 500 has an angle of slope S″ with the vertical line at the inner peripheral edge 512 at the first open end 522, and the inner surface 504 has an angle of slope S′″ with the vertical line at the inner peripheral edge 516 at the second open end 524. It may be noted that for the suturing ring 500, the angle of slope S″ and the angle of slope S′″ are different with respect to each other. In an example implementation, S″ and S′″ is in a range of about 10 degrees to 80 degrees. Different angles of slope of the inner surface 504 at the first open end 522 and the second open end 524 provides a variation in hemodynamic characteristics during inflow and outflow of blood through the suturing ring 500 which forms a valve housing of a prosthetic heart valve.

The suturing ring 500 has a substantially uniform annular thickness from the first open end 522 to the second open end 524. In an example implementation, the annular thickness is in a range of about 0.3 mm to about 1.5 mm.

It may be noted the suturing ring 500 has suture holes or suture-receiving passages 520, similar to the suture-receiving passages 420 of the suturing ring 400 as shown in FIGS. 4a and 4b.

The suturing ring 500 may be manufactured by following a procedure similar to the procedures for manufacturing the suturing ring 400. For manufacturing the suturing ring 500 asymmetrical about the mid-circumferential plane, two mandrels with an axisymmetric shape that directly corresponds to the inner surface profile of the suture ring having non-identical diameters are used.

In an example implementation, the suturing rings 100, 400, and 500 may have a varying annular thickness towards the first end and the second end. The varying annular thickness towards the first open end and the second open end may result due to stretching of the metal during the metal forming procedures carried out while manufacturing the suturing ring. As a result of this stretch, the annular thickness of the suturing ring may be reduced towards the first end and the second end as compared to the annular thickness at the mid-circumferential plane. The suturing ring with reduced annular thickness at the first and second ends provides improved hemodynamic blood flow, reduction in turbulence in blood flow and thereby reduces risks of thrombosis, and facilitates simpler implant of the prosthetic valve within the human body during a surgical procedure.

Although implementations for suturing rings and method of manufacturing suturing rings are described, it is to be understood that the present subject matter is not necessarily limited to the specific features described herein. Rather, the specific features are disclosed as example implementations of suturing rings and methods of manufacturing suturing rings.

I/We claim:

1. A method of manufacturing a suturing ring for a prosthetic heart valve, the method comprising:
forming a plurality of suture holes through a rectangular metal sheet along a length of the metal sheet, wherein the metal sheet has a substantially constant thickness throughout the length of the metal sheet;
rolling the rectangular metal sheet along the length until two ends of the metal sheet abut with each other;
welding the two ends of the metal sheet to form a metallic tube; and
reshaping the metallic tube using a forming tool to obtain a suturing ring having an inner surface with a substantially convex profile, such that an inner diameter at an inner peripheral edge at a first open end and an inner diameter at an inner peripheral edge at a second open end of the suturing ring is greater than an inner diameter at a mid-circumferential plane of the suturing ring.

2. The method as claimed in claim 1, wherein the forming the plurality of suture holes comprises:
forming a first set of suture holes through the metal sheet between a central longitudinal axis of the metal sheet and a first linear edge of the metal sheet; and
forming a second set of suture holes through the metal sheet between the central longitudinal axis of the metal sheet and a second linear edge of the metal sheet.

3. The method as claimed in claim 1, wherein the reshaping comprises:
pressing a first mandrel with an axisymmetric shape that directly corresponds to the inner surface profile of the suture ring against the metallic tube from the first open end to exert an outward force on the inner surface of the metallic tube; and
pressing a second mandrel with an axisymmetric shape that directly corresponds to the inner surface profile of the suture ring against the metallic tube from the second open end to exert an outward force on the inner surface of the metallic tube.

4. The method as claimed in claim 3, wherein the first mandrel and the second mandrel have identical diameters, and wherein the suturing ring is symmetrical about the mid-circumferential plane.

5. The method as claimed in claim 3, wherein the first mandrel and the second mandrel have non-identical diameters, and wherein the suturing ring is asymmetrical about the mid-circumferential plane.

6. A method of manufacturing a suturing ring for a prosthetic heart valve, the method comprising:
forming, by one of punching and machining, a plurality of suture holes passing from an inner surface to an outer surface of a metallic tube, wherein the metallic tube has a substantially constant annular thickness from a first open end to a second open end of the metallic tube; and
reshaping the metallic tube using a forming tool to obtain a suturing ring having an inner surface with a substantially convex profile, such that an inner diameter at an inner peripheral edge at a first open end of the suturing ring and an inner diameter at an inner peripheral edge at a second open end of the suturing ring is greater than an inner diameter at a mid-circumferential plane of the suturing ring.

7. The method as claimed in claim 6, wherein the forming the plurality of suture holes comprises:
forming a first set of suture holes in the metallic tube between a mid-circumferential plane of the metallic tube and an inner peripheral edge at the first open end of the metallic tube; and
forming a second set of suture holes in the metallic tube between the mid-circumferential plane of the metallic tube and an inner peripheral edge at the second open end of the metallic tube.

8. The method as claimed in claim 6, wherein the reshaping comprises:
pressing a first mandrel with an axisymmetric shape that directly corresponds to the inner surface profile of the suture ring against the metallic tube from the first open end to exert an outward force on the inner surface of the metallic tube; and
pressing a second mandrel with an axisymmetric shape that directly corresponds to the inner surface profile of the suture ring against the metallic tube from the second open end to exert an outward force on the inner surface of the metallic tube.

9. The method as claimed in claim 8, wherein the suturing ring obtained after the reshaping is symmetrical about the mid-circumferential plane when the first mandrel and the second mandrel have identical diameters, and wherein the suturing ring obtained after the reshaping is asymmetrical about the mid-circumferential plane when the first mandrel and the second mandrel have non-identical diameters.

10. The method as claimed in claim 6, further comprising:
rolling a rectangular metal sheet of substantially constant thickness along a length of the metal sheet until two ends of the metal sheet abut with each other; and
welding the two ends of the metal sheet to form the metallic tube.

11. A suturing ring forming a valve housing of a prosthetic heart valve, wherein the suturing ring has an inner surface having a substantially convex profile, wherein the suturing ring has an inner diameter at an inner peripheral edge at a first open end and at an inner peripheral edge at a second open end of the suturing ring greater than an inner diameter at a mid-circumferential plane of the suturing ring, and wherein the suturing ring has a substantially constant annular thickness from the first open end to the second open end.

12. The suturing ring as claimed in claim 11, wherein the inner surface of the suturing ring has a planar portion substantially perpendicular to the mid-circumferential plane of the suturing ring, and wherein the planar portion of the inner surface extends up to a first distance from the mid-circumferential plane towards the first open end and up to a second distance from the mid-circumferential plane towards the second open end.

13. The suturing ring as claimed in claim 11, wherein the suturing ring is symmetrical about the mid-circumferential plane.

14. The suturing ring as claimed in claim 11, wherein the suturing ring is asymmetrical about the mid-circumferential plane.

15. The suturing ring as claimed in claim 11, wherein the inner surface at the inner peripheral edge at the first open end and at the inner peripheral edge at the second open end have an angle of slope in a range from about 10 degrees to about 80 degrees.

16. The suturing ring as claimed in claim 11, wherein the suturing ring comprises a plurality of suture holes from the inner surface to an outer surface of the suturing ring.

17. The suturing ring as claimed in claim 11, wherein the constant annular thickness of the suturing ring is in a range from about 0.3 mm to about 1.5 mm.

18. The suturing ring as claimed in claim 11, wherein the inner diameter of the suturing ring at the mid-circumferential plane is in a range from about 12 mm to about 32 mm.

19. The suturing ring as claimed in claim 11, wherein the inner diameter of the suturing ring at the first open end and the second open end is in a range from about 15 mm to about 35 mm.

20. The suturing ring as claimed in claim 11, wherein the first open end and the second open end are separated by a linear distance in a range from about 4.5 mm to about 10.5 mm.
